(19) United States
(12) Patent Application Publication
(10) Pub. No.: US 2019/0125932 A1
(43) Pub. Date: May 2, 2019

(54) PREVENTING BLOOD CLOT FORMATION, CALCIFICATION AND/OR PLAQUE FORMATION ON BLOOD CONTACT SURFACE(S)

(71) Applicant: Cal-X Stars Business Accelerator, Inc., Playa Vista, CA (US)

(72) Inventors: Howard J. Leonhardt, Playa Vista, CA (US); Jeff Donofrio, Anaheim, CA (US)

(21) Appl. No.: 16/172,405

(22) Filed: Oct. 26, 2018

Related U.S. Application Data
(60) Provisional application No. 62/577,395, filed on Oct. 26, 2017.

(51) Int. Cl. A61L 27/50; A61L 27/06; A61L 33/02; A61L 27/36

(52) U.S. Cl. CPC A61L 27/507; A61L 27/06; A61L 27/047; A61L 27/3625; A61L 33/022

(57) ABSTRACT

Described is a device for preventing thrombosis formation on surfaces of a blood contact device. The device may first non-invasively scan the blood contact device and determines the highest risk thrombosis points. The device then, preferably starting with the highest risk location, delivers a succession of harmonic vibration signals or electromagnetic signals non-invasively so as to prevent clot formation at each stagnation high risk point of the blood contact device (e.g., harmonic resonance). This resonant vibration calibration tuning information is stored in an associated microprocessor. The signals are then delivered, based upon the stored information, in a loop from the signal generator, usually on a belt outside the patient, to each stagnation point in sequence from highest risk of thrombosis to lowest; again and again repeated. By delivering such energy to the blood contact device stagnation points, initiation of thrombosis formation is prevented, thus preventing the accumulation of thrombosis to a dangerous risk level for stroke, pulmonary embolism, and/or other blood clot induced ailments. This device may be used to prevent and/or treat blood clot, plaque, and/or calcification formation on any blood contact surfaces including living surfaces such as heart valves.

### CROSS-REFERENCE TO RELATED APPLICATION

This application claims the benefit under 35 U.S.C. § 119 to U.S. provisional patent application 62/577,395, filed on Oct. 26, 2017, the contents of which are incorporated herein by this reference.

### FIELD

This application relates generally to the field of medical devices and associated methods of using them, and more specifically to methods and devices for treating surface(s) and devices that come into contact with blood. In certain embodiments, select vibratory or electromagnetic signals are delivered to the surface(s) so as to attain, e.g., a harmonic frequency for a specific treatment surface of the device and associated methods. The resulting system utilizes optimized signals to prevent and/or treat the formation of blood clots, calcification, and/or plaque.

### BACKGROUND

Blood clots, calcification and plaque form on blood contact surfaces within the human body and within blood contact medical devices. These unwanted build ups can cause strokes, embolisms, and heart attacks causing the loss of life, brain or body functions, and further reduce quality of life.

Reportedly, every five minutes someone in the United States dies from a blood clot. Vascular Disease Foundation. “Every five minutes someone dies from a blood clot or deep vein thrombosis.” ScienceDaily, 5 Mar. 2011. Blood clot formation on blood contact surfaces is a major cause of death and debilitating damage to brain, heart and lung function. Approximately 300,000 deaths are from blood clots annually in the US alone. Blood clot formation is believed to be the leading cause of failure of circulatory assist devices and heart pumps. It is a primary source of serious adverse effects associated with all blood contact devices such as heart valves and long term use catheters. It is the primary cause need for the use of blood thinning drugs which have dangerous side effects and can cause death themselves from un-controlled bleeding.

Kormos et al. “Left Ventricular Assist Device Malfunctions: It's More Than Just The Pump”, CIRCULATIONAHA.117.027360, originally published Jul. 3, 2017 (doi.org/10.1161/CIRCULATIONAHA.117.027360) reported that 26% of patients had pump blood clot thrombosis with Heartmate II after 3 years. Heartmate II (Thoratec Corporation) is a heart pump called a left ventricular assist device (LVAD), which was designed to assist the left side of the heart to pump the blood a body needs. Only 74% of the HeartMate II patients were free of pump thrombosis at 3 years compared with 90% of the HVAD patients. Freedom from failure of the integrated driveline was 79% at 3 years for the HMII but 100% for the HVAD (log-rank p<0.02). This calculates out to 26% of patients had pump blood clot thrombosis with HeartMate II.

U.S. Pat. No. 5,788,668 to Leonhardt, Aug. 4, 1998, the contents of which are incorporated herein by this reference in its entirety, describes a method whereby a programmable signal source produces a desired output signal that is transferred by a conduit means or conducting means into a patient by percutaneous venous insertion. The output signal is either vibrational or electrical. If vibrational, the conduit means or one or more transducers radiates the output signal into the treatment site within a patient. If the output signal is electrical, one or more transducers receive the output signal and convert the output signal into vibration and then radiate it into the treatment site within a patient. The treatment site is the location of a catheter or other intravenous device, residing within the patient for the purposes of gas exchange in the blood stream or for other long-term treatment. The presence of the vibration increases the efficiency of intravenous gas exchanging devices significantly, and prevents clot formation on the surface of intravenous devices. The programmable output source includes signal conditioning allowing the output signal to be manipulated so as to best accommodate whatever particular catheter or device is being employed. The increase in efficiency of the catheter or device resulting from the vibration correlates into safer patient treatment and longer life for the catheter or device employed. Leonhardt however utilized a vibrating coiled guide wire inside a catheter with one signal (attached it to an engraving vibrator) and was primarily for gas exchange improvement in hollow fibers.

In the past, people of skill in the art have tried to create a negative charge on surfaces by coating a metal surface with nanomaterials of TiO₂ and SiO₂ or ZnO. For example, PCT International Publication WO2015189716A1 (“Thrombosis resistant mechanical”), Dec. 17, 2015, the contents of which are incorporated herein by this reference, describes mechanical heart valves that use nanotechnology through the addition of nanomaterials of TiO₂ and SiO₂ or ZnO to all surfaces of the mechanical valve (including leaflets and their surrounding ring). These nanomaterials induce “superhydrophobia,” and establish a negative charge. See also, U.S. Pat. No. 4,038,702 to Sawyer (Sep. 21, 1973), the contents of which are incorporated herein by this reference, for “Electrochemical and chemical methods for production of non-thrombogenic metal heart valves” for further surface treatments.

Unfortunately however, when ingested, nanomaterials of TiO₂ and SiO₂ or ZnO can also have toxic side effects in blood including cerebral function loss. See, e.g., L Tian et al. “Neurotoxicity induced by zinc oxide nanoparticles: age-related differences and interaction” Nature Scientific Reports, 2015, 5:16117|doi: 10.1038/srep16117; Fadeel et al. Adverse Effects of Engineered Nanomaterials: Exposure, Toxicology, and Impact on Human Health (2017); Ghosh et al. “Cytotoxic, genotoxic and the hemolytic effect of titanium dioxide (TiO₂) nanoparticles on human erythrocyte and lymphocyte cells in vitro (2013), doi.org/10.1002/jat.2863; and Setyawati M I, et al. Small 2015 July; 11(28):3458-68. doi: 10.1002/smll.201403232. Epub 2015 Apr. 22.

### BRIEF SUMMARY

Described, among other things, is a device that includes a sensor that detects harmonic frequency and/or electromagnetic energy and recognizes resonance and/or ionic charge on a blood contact surface, a microprocessor for analyzing data from the sensor, and an emitter of sound, ultrasound, and/or electromagnetic energy associated with said microprocessor that can create and focus sound, ultrasound, and/or electromagnetic energy onto the blood contact surface. Typically, the sensor device detects, e.g., harmonic frequency, and the device reads and then the microprocessor thereof custom tunes the appropriate harmonic frequency to prevent and/or treat blood clots, calcification, and/or plaque formation on the blood contact surface. Typically the device includes a strap or other means that affixes the device to a living subject in which the blood contact surface has been implanted.

The described devices may be used with blood arteries, veins, hearts, heart valves, pacemakers, and all blood contact devices such as left ventricle assist devices.

Also, typically, the device further includes an associated resonant calibration switch for triggering the device to scan a blood contact device having the blood contact surface to determine the highest risk stagnation point(s). In operation, such a device delivers a sequence of signals to each stagnation point until resonant harmonic vibration for the blood contact surface is reached so that blood clots do not affix to the blood contact surface.

In certain embodiments, the emitter emits electromagnetic radiation that applies a selected electromagnetic radiation to the blood contact surface, and the microprocessor controls delivery of the electromagnetic radiation from the emitter to the blood contact surface so as to prevent the beginning of blood clot formation, calcification and/or plaque formation on the blood contact surface.

In use, the device typically applies an appropriate harmonic frequency or electromagnetic energy to a blood contact surface within a living subject so as to prevent and/or dislodge blood clot(s), calcification(s), and/or plaque formation(s) on the blood contact surface.

In certain embodiments, described is a harmonic vibration energy device (or “vibrational harmonic resonant energy device”) that applies an appropriate harmonic frequency to a blood contact surface to prevent the beginning formation of a blood clot formation, calcification, and/or plaque formation on the blood contact surface. Described is a wireless (e.g., acoustic) harmonic-tuned vibration device that has a different signal for every high risk stagnation point on a blood contact surface. The described device preferably records the signals for reaching resonance for each stagnation point on the blood contact surface and plays back the signals in cycles on loop.

In certain “vibratory” embodiments, the harmonic vibration energy device first measures an appropriate harmonic frequency to prevent the beginning formation of a blood clot formation, calcification, and/or plaque formation on a blood contact surface of a device, and then custom tunes and applies the appropriate harmonic frequency to the blood contact surface to prevent the initial formation of a blood clot formation, calcification, and/or plaque formation on the blood contact surface.

The harmonic vibration device customizes a vibration signal for each surface stagnation point of a structure prone to blood clot formation, calcification, and/or plaque aggregation, and prevents the first particles from adhering, thus stopping the progressive build up before it begins. The device may also be used to treat blood clot formation, calcification, and/or plaque aggregation.

Prior art devices delivered a single signal that was not tuned to reach a harmonic frequency for the specific treatment surface. Also prior art devices utilized much stronger vibratory signals in an attempt to break up build ups, but were not intended to prevent their initiation in the first place. Previous devices also failed to tune to the right frequency for the specific treatment surface.

The devices described herein prevent the starting point of buildup. No other device is known to customize the vibrational signal to reach harmonic frequency for the specific treatment surface and cycle through a variety of signals when needed for multi-surface applications.

Also described is a method of preventing the formation of a blood clot, calcification, and/or plaque on a blood contact surface, the method comprising: applying a harmonic frequency to a blood contact surface to prevent the beginning formation of a blood clot formation, calcification, and/or plaque formation on the blood contact surface. In such a method, a wireless harmonic-tuned vibration device may be used to apply the harmonic frequency to the blood contact surface. The wireless harmonic-tuned vibration device preferably has a custom signal for multiple high risk stagnation points on the blood contact surface. The method may further include recording the signals for reaching resonance for each stagnation point on the blood contact surface. It may also include playing the signal back in cycles on loop.

Further described is a method of preventing the formation of a blood clot, calcification, and/or plaque on a blood contact surface of a subject or a device, the method comprising: first measuring an appropriate harmonic frequency to prevent the beginning formation of a blood clot formation, calcification, and/or plaque formation on a blood contact surface of a device; and then custom tuning and applying an appropriate harmonic frequency to the blood contact surface to prevent the initial formation of a blood clot formation, calcification, and/or plaque formation on the blood contact surface. In such a method, a wireless harmonic-tuned vibration device that provides a custom signal for multiple high risk stagnation points on the blood contact surface may be utilized. Such a method may further include recording signals for reaching resonance for each stagnation point on the blood contact surface. Also, the method may further include playing back the signal in cycles on loop. The device with the blood contact service may be selected, e.g., from the group consisting of a heart valve, pacemaker, and a left ventricle assist device.

A preferred vibrational harmonic resonant energy device scans internal blood contact devices and organs to identify (particularly high risk) stagnation points for blood clot formation. It starts with the highest risk points and to them delivers vibrational acoustic energy until harmonic resonance is reached. The tuning process of the device then moves on to the second highest risk stagnation point and continues until all high risk areas are addressed. The microprocessor of the device then cycles through delivering harmonic resonant vibrational energy to each high risk point to reduce the risk of blood clot formations even beginning to form. This device is designed to work on the principle that if a blood clots (or clots) cannot begin to form in the first place that it cannot get large enough to cause problems.

In certain embodiments, the elasticity of natural tissues are improved so that they vibrate better.

Also described is an electromagnetic surface treatment device that applies an appropriate electromagnetic radiation to a blood contact surface to, e.g., prevent the beginning formation of a blood clot formation, calcification, and/or plaque formation on the blood contact surface. Also described are apparatus and methods for utilizing electromagnetic energy to change the surface properties of, for example, surfaces on an implantable or ex vivo device(s) to prevent the formation of blood clots and calcification thereon. In certain electromagnetic embodiments, the described apparatus and methods utilize the opposite polarity to encourage the body to attack cancer tumors (e.g., negative to repel and positive to attract.)

The described electromagnetic surface treatment apparatus and methods may be used with metal and animal heart valves and with implantable circulatory assist pumps (which are both prone to blood clots and calcification). Such apparatus and methods can prevent blood clots and calcification with reduced side effects.

In certain electromagnetic embodiments (e.g., when the surfaces of the implantable or ex vivo device are plastic), the plastic surface may be coated with a metallic or otherwise electrically conductive coating, and then a voltage applied to the coating (e.g., from a power source such as a battery). Such a coating may be applied by, e.g., electrostatic spraying of the metallic or electrically conductive material onto the surface, for example, before implantation of the device into the subject. Alternatively, the surface may be coated with molten metal.

In certain electromagnetic embodiments, a liquid having a negative static electrical charge can be coated onto the plastic surface(s), causing the surface(s) to become, e.g., negatively charged. Since like static electrical charges repel one another, the negative static electrically charged liquid coating repels negatively charged particles. In certain electromagnetic embodiments, the liquid can be coated onto the surface and then rubbed to a thin film with an appropriate sheet or polisher. Compare, US 20040185178 A1 to Rood (Mar. 20, 2003), the contents of which are incorporated herein by this reference.

In certain electromagnetic embodiments, plasma immersion ion implantation and deposition can be used to modify the surface(s) appropriately. Compare, Lu et al. “Surface modification of biomaterials using plasma immersion ion implantation and deposition”, Interface Focus, 2012 Jun. 6; 2(3): 325-336, doi: 10.1098/rsfs.2012.0003, the contents of which are incorporated herein by this reference.

In certain electromagnetic embodiments, the metallic or other conductive surface alone may be sufficient to repel clots in the particular in vivo or ex vivo environment.

In certain electromagnetic embodiments, the polarity of the applied voltage can be momentarily switched to positive, for example, so that positively charged biomaterial surface can exert an antimicrobial effect on any adhering Gram-negative bacteria. Compare, Gottenbos et al. “Antimicrobial effects of positively charged surface on adhering Gram-positive and Gram-negative bacteria,” Journal of Antimicrobial Chemotherapy, 48(1):7-13—August 2001, doi: 10.1093/jac/48.1.7 the contents of which are incorporated herein by this reference.

In certain embodiments, the electromagnetic surface treatment device first measures an appropriate electromagnetic radiation to prevent the beginning formation of a blood clot formation, calcification, and/or plaque formation on a blood contact surface of a device, and then custom tunes and applies the appropriate electromagnetic radiation to the blood contact surface to prevent the initial formation of a blood clot formation, calcification, and/or plaque formation on the blood contact surface. The device may also be used to treat blood clot formation, calcification, and/or plaque aggregation.

The electromagnetic surface treatment device may also customize an electromagnetic signal for each surface stagnation point of a structure prone to blood clot formation, calcification, and/or plaque aggregation, and prevent the first particles from adhering, thus stopping the progressive build up before it begins.

Such a device prevents the starting point of buildup. No other device is known to customize the electromagnetic signal to reach electromagnetic radiation for the specific treatment surface and cycle through a variety of signals when needed for multi-surface applications.

The described electromagnetic surface treatment device may be used with blood arteries, veins, hearts, heart valves, pacemakers, and all blood contact devices such as left ventricle assist devices.

Also described is a method of preventing the formation of a blood clot, calcification, and/or plaque on a blood contact surface, the method comprising: applying electromagnetic radiation to a blood contact surface to prevent the beginning formation of a blood clot formation, calcification, and/or plaque formation on the blood contact surface.

In some embodiments, a wireless device may be used to apply the electromagnetic radiation to the blood contact surface. The wireless device may have a custom signal for multiple high risk stagnation points on the blood contact surface. The method may further include recording the signals for reaching resonance for each stagnation point on the blood contact surface. It may also include playing the signal back in cycles on loop.

Further described is a method of preventing the formation of a blood clot, calcification, and/or plaque on a blood contact surface of a subject or a device, the method comprising: first measuring an appropriate electromagnetic radiation to prevent the beginning formation of a blood clot formation, calcification, and/or plaque formation on a blood contact surface of a device; and then custom tuning and applying an appropriate electromagnetic radiation to the blood contact surface to prevent the initial formation of a blood clot formation, calcification, and/or plaque formation on the blood contact surface. In such a method, a wireless device that provides a custom signal for multiple high risk stagnation points on the blood contact surface may be utilized. Such a method may further include recording signals for reaching resonance for each stagnation point on the blood contact surface. Also, the method may further include playing back the signal in cycles on loop. An implantable device with the blood contact service may be selected, e.g., from the group consisting of a heart valve, pacemaker, and a left ventricle assist device.

A preferred electromagnetic surface treatment device scans internal blood contact devices and organs to identify (particularly high risk) stagnation points for blood clot formation. It starts with the highest risk points and to them delivers electromagnetic radiation. The tuning process of the device then moves on to the second highest risk stagnation point and continues until all high risk areas are addressed. The microprocessor of the device then cycles through delivering electromagnetic energy to each high risk point to reduce the risk of blood clot formations even beginning to form. This device is designed to work on the principle that if a blood clots (or clots) cannot begin to form in the first place then it cannot get large enough to cause a problem.

In use, the device may be mounted on VELCRO™ or, e.g., an elastic or other belt, strap, or tie that wraps around the region of the subject to be treated at the time. Alternatively, a “vest” could be used (not shown). Communications to a subject's cellular telephone instructs him or her where to position the belt for optimization of results at particular times.

In use, the device may be mounted on, e.g., an elastic belt that wraps around the region of the subject to be treated at the time. Communications to a subject's cellular telephone instructs him or her where to position the belt for optimization of results at particular times.

The device may be used as a complementary support technology to a circulatory assist product and other blood contact devices especially those intended for chronic use. Time lapse photography studies show that nearly every blood clot begins by an initial attachment point, which then serves as a point of aggregation and accumulation until the blood clot becomes large enough to become dangerous. Vibrational harmonic resonant energy when applied right can stop this process from beginning.

Described is an electromagnetic surface treatment device for preventing thrombosis formation on surfaces of blood contact devices. The device may first non-invasively scan the blood contact device and determine the highest risk thrombosis points. Such a device then, preferably starting with the highest risk location, delivers a succession of electromagnetic signals non-invasively for each stagnation high risk point of the blood contact device. This information is stored in an associated microprocessor. The signals are then delivered, based upon the stored information, in a loop from the signal generator, usually on a belt outside the patient, to each stagnation point in sequence from highest risk of thrombosis to lowest; again and again repeated. By delivering electromagnetic energy to the blood contact device stagnation points, initiation of thrombosis formation is prevented, thus preventing the accumulation of thrombosis to a dangerous risk level for stroke, pulmonary embolism, and/or other blood clot induced ailments. This device may be used to prevent blood clot, plaque, and/or calcification formation on any blood contact surfaces including living surfaces such as heart valves.

### BRIEF DESCRIPTION OF THE DRAWINGS

FIG. 1 depicts a device that includes (a) a harmonic vibration signal generator and (b) a harmonic vibration microprocessor analyzer mounted on an elastic belt wrapped around a subject's leg, with communications to a subject's cellular telephone instructing the subject, e.g., where to position the belt for optimization of results at particular times.

FIG. 2 depicts a device that includes (a) a harmonic vibration signal generator and (b) a harmonic vibration microprocessor analyzer mounted on an elastic belt about the subject's chest, with communications to a subject's cellular telephone instructing the subject, e.g., where to position the belt for optimization of results at particular times.

FIG. 3 depicts a device that includes (a) an electromagnetic radiation generator and (b) a microprocessor analyzer mounted on an elastic belt wrapped around a subject's leg, with communications to a subject's cellular telephone instructing the subject, e.g., where to position the belt for optimization of results at particular times.

FIG. 4 depicts a device that includes (a) an electromagnetic radiation generator and (b) a microprocessor analyzer mounted on an elastic belt about the subject's chest, with communications to a subject's cellular telephone instructing the subject, e.g., where to position the belt for optimization of results at particular times.

### DETAILED DESCRIPTION

In certain embodiments, described is a vibrational device for preventing thrombosis formation on blood contact surfaces. Blood clots, calcification and plaque form on blood contact surfaces within the human body and within blood contact medical devices. These unwanted build ups can cause strokes, embolisms, and heart attacks causing loss of life, brain or body functions and quality of life. The devices described herein prevent the formation of blood clot formation, calcification, and/or plaque formation on blood contact surfaces.

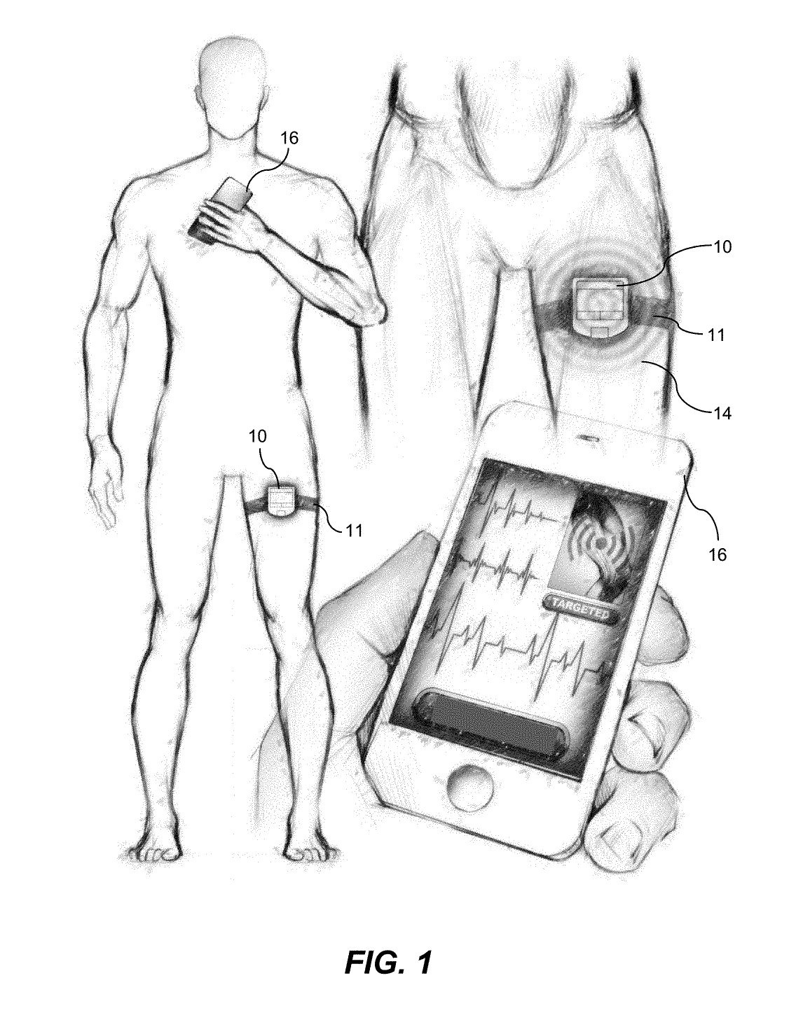

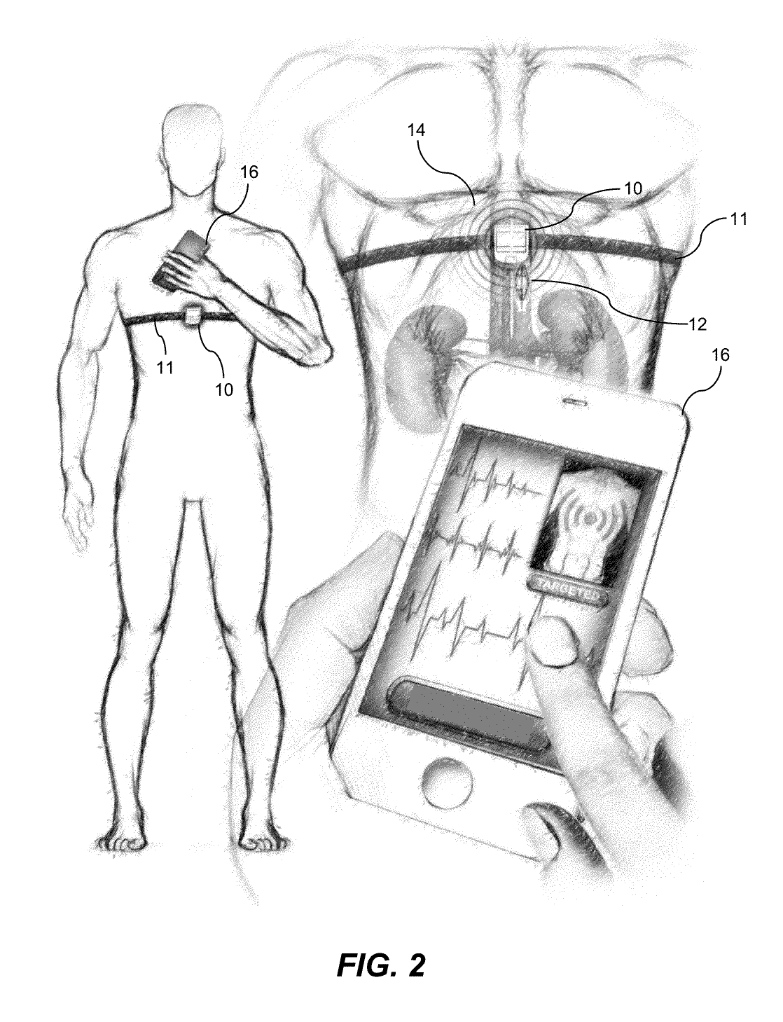

The vibrational device 10 (FIGS. 1 and 2) typically includes a sensor, a harmonic vibration signal generator or sensor, and a harmonic vibration microprocessor analyzer optionally mounted or affixed, e.g., by a belt 11 to a subject. In some embodiments, it lacks (or disables or ignores) the sensor, and predetermined signals may then be used to achieve resonance if desired. The harmonic vibration signal generator can be any device (e.g., a “speaker”) able to create and deliver and/or target acoustic or other vibrational energy signals to a desired location (e.g., focused or directed sound akin to a focalized tuning fork at stagnation points associated with the blood contact device). The harmonic vibration microprocessor analyzer can be any microprocessor with associated memory able to store information that can be retrieved on demand and analyzed with appropriate software. The harmonic vibration microprocessor analyzer is preferably programmed to recognize resonance at the blood contact surface. The device typically includes an associated resonant calibration switch for triggering the device to scan, e.g., a blood contact device 12 to determine the highest risk stagnation points, and then to deliver a sequence of signals 14 at each stagnation point until resonant harmonic vibration is reached. The device 10 communications with, e.g., a subject or health care provider's cellular telephone 16, e.g., by BLUE TOOTH™ technology, instructing the subject, e.g., where to best position the belt for optimization of results at particular times Afterward, the operator may, e.g., then actuate a run thrombosis prevention loop program.

The harmonic vibration device preferably utilizes a custom vibration signal for each surface stagnation point prone to blood clot formation, calcification, and/or plaque aggregation and stops the initial particles from adhering; thus stopping the progressive build up before it starts. Previous devices failed to tune to the correct frequency for the specific treatment surface. The instant device customs tunes (at the correct frequency) to reach harmonic vibration of the particular stagnation prone surface, thus preventing the beginning of buildup and blood clot formation, calcification, and/or plaque aggregation.

Such a device preferably customizes the vibrational signal to reach harmonic frequency for the specific treatment surface and cycles through a variety of signals when needed for multi-surface applications.

The harmonic vibrational device preferably first non-invasively scans a blood contact device and determines the highest risk thrombosis points in a system or blood contact device. See, e.g., the incorporated Tosi et al. “Vibrational spectroscopy as a supporting technique in clinical diagnosis and prognosis of atherosclerotic carotid plaques: a review”, Anal Quant Cytopathol Histpathol. 2012 August; 34(4):214-32 and Nakazawa et al. “Vibration assessment for thrombus formation in the centrifugal pump” Artif Organs. 1997 April; 21(4):318-22.

The harmonic vibrational device then, starting with the highest risk location, delivers a succession of harmonic vibration signals non-invasively until harmonic resonance is reached for each stagnation high risk point of the blood contact device. This resonant vibration calibration tuning information is stored in a microprocessor preferably associated with the harmonic vibrational device. The signals may then be delivered, based upon the stored information, in a loop from the signal generator (e.g., on a belt outside the patient), to each identified stagnation point, preferably in sequence from highest risk of thrombosis to lowest risk, which is again and again repeated.

By delivering harmonic resonant vibrational energy to the blood contact device stagnation points, the initiation of thrombosis formation is prevented, thus preventing the accumulation of thrombosis to a dangerous risk level for stroke, pulmonary embolism, and/or other blood clot induced ailments. The vibrational device may be used to prevent blood clot(s), plaque(s), and/or calcification(s) on any blood contact surface, including living surfaces such as heart valves.

The device preferably customs tunes—at the correct frequency—to reach harmonic vibration of the particular stagnation prone surface thus preventing the beginning of buildup.

In manufacturing and testing, the device is first preferably mounted in a manner similar to the way it will be put to use with a subject. A vibrator, which preferably comprises a variable speed eccentric motor, is mounted on (or otherwise associated with) the device and coupled to a control electronics package. A vibration transducer is likewise mounted on device and provides an electrical output to the electronics package as a function of amplitude of device vibration. The electronics package includes a knob or other suitable control means for selectively varying frequency of vibration applied to the device by a motor, a gauge or other suitable readout for indicating frequency of vibration to an operator, and an output coupled to a recorder for providing on, e.g., an X-Y plotter having the frequency response characteristics of device recorded thereon.

In a particular aspect, a method of analyzing a device comprises applying mechanical cyclic vibration energy to a the device over a test frequency range; monitoring damping effects of energy flowing into the device as a function of frequency and identifying a plurality of orders of harmonic vibration absorption peaks, each consisting of a plurality of vibration absorption resonant peaks; and then applying mechanical cyclic vibration energy to the device for an extended period of time at fixed frequency corresponding to a sub-harmonic frequency of one of the harmonic peaks.

In such a method, monitoring damping effects of energy flowing into the device as a function of frequency and identifying a plurality of orders of harmonic vibration absorption peaks may comprise mounting a vibration transducer on the device to provide an electrical output signal as a function of vibration amplitude, and damping response of the transducer to mechanical vibration such that the output varies as a function of harmonic groups of vibration resonant peaks.

The method may also include, before applying mechanical cyclic vibration energy, selecting the fixed frequency as a function of composition of the device. In such a method, this may comprise selecting a part of a particular order of harmonics from among the plurality of orders as a function of composition of the device, and identifying a sub-harmonic frequency associated with the part of a particular order of harmonics and corresponding to a vibration amplitude equal to approximately one-third of maximum vibration amplitude of the part of a particular order.

In another aspect, a method of analyzing a part of a device includes applying mechanical cyclic vibration energy to the part of a device over a test frequency range, monitoring damping effects of energy flowing into the part of a device as a function of frequency by mounting a vibration transducer on the part of a device to provide an electrical output signal as a function of vibration amplitude and damping response of the transducer to mechanical vibrations such that the output varies as a function of harmonic groups of vibration resonant peaks, identifying at least one peak of harmonic vibration absorption consisting of a plurality of vibration absorption resonant peaks, and then applying mechanical cyclic vibration energy to the part of a device for an extended period of time at fixed frequency corresponding to a sub-harmonic frequency of the at least one harmonic peak.

In such a method, the method can further include monitoring the damping effects while applying energy, identifying any changes in harmonic frequency of the one peak, and reselecting the one peak as a function of the changes identified.

U.S. Pat. No. 7,824,358 B2 to Cotter et al., Nov. 2, 2010 (the contents of which are incorporated by this reference in its entirety), discloses a heart assist device connection system comprising an inflow connector in fluid-tight communication with an inflow section of a heart assist device. The connector is configured to be releasably connected to an inlet extension inserted into a patient's ventricle. The connector has one or more recesses configured to match a protrusion on the inlet extension. The system also comprises an outflow connector in fluid communication with an outflow section of the heart assist device. The outflow connector is configured to be releasably connected to a conduit attached-to the patient's vasculature.

The natural vibration (“harmonic”) frequencies of a surface may be determined (or estimated) based upon its length, Young's modulus for the material present, mass density, and structure of the surface. There are natural frequencies for both longitudinal and compressional waves, which generally have different values. A standard mathematical technique exists for this, which may be implemented with, e.g., a software package.

The natural vibration frequencies will depend somewhat on, e.g., whether the device containing the surface is secured or free. If secured, the device needs to be done so firmly to a very strong, heavy object or the resonances will be “lossy.” If unsecured, one can set the device on springs or hang it from, e.g., a bungee cord.

There are a variety of techniques to apply vibration to the device. One can “hit” it, e.g., utilizing a piezoelectric hammer that measures how much force was applied. One can push on it with a probe attached to a piezoelectric or electromagnetic pusher. If the surface is magnetic or can have a magnet attached, one can use an electromagnet. One can also bombard it with sound (e.g., via a speaker system). If nothing else is suitable, one might put the device near a high voltage and push it electrostatically.

To sense the vibration, there are many contacting and non-contacting sensors, including magnetic, capacitive/electrostatic and optical. For larger objects, an accelerometer may be used.

In order to find frequencies in the output, one may utilize a spectrum analyzer, which generates waveforms suitable for driving the forcer, takes the Fourier transform of both output and input, and divides to produce a transfer function. Peaks corresponding to the resonances will result.

Potential applications for the device include applying vibratory energy to heart implants, left ventricle assist devices (which are place closer to the heart), in place heart valves, coronary arteries and coronary stents within coronary arteries that wrap around the heart, in the legs (see, e.g., FIG. 1, but also including having multiple devices up on down the legs separated about 12 inches apart from the upper thigh to the ankles), hair growth stimulation, acceleration of tooth growth, treatment of erectile dysfunction recovery, and/or brain function recovery.

For extracorporeal devices, vibration may be administered, e.g., by a commercially available vibrator (e.g., available from Hitachi) tuned to the particular harmonic frequency. Alternatively, a variable speed eccentric motor may be mounted to the device.

As used herein, electromagnetic radiation includes radio waves, microwaves, infrared, light, visible light, ultraviolet, X-rays, and gamma rays. Generators and/or emitters of each electromagnetic radiation is known to those of ordinary skill in the art.

Also described herein is an electromagnetic radiation generating device for preventing thrombosis formation on blood contact surfaces. Blood clots, calcification and plaque form on blood contact surfaces within the human body and within blood contact medical devices. These unwanted build ups can cause strokes, embolisms and heart attacks causing loss of life, brain or body functions and quality of life. The electromagnetic surface treatment device prevents the formation of blood clot formation, calcification, and/or plaque formation on blood contact surfaces, including the starting point of buildup. While not intending to be bound by theory, it is believed that the applied electromagnetic radiation changes surface ions on the blood contact surface to be repulsive to the materials forming, e.g., the clot.

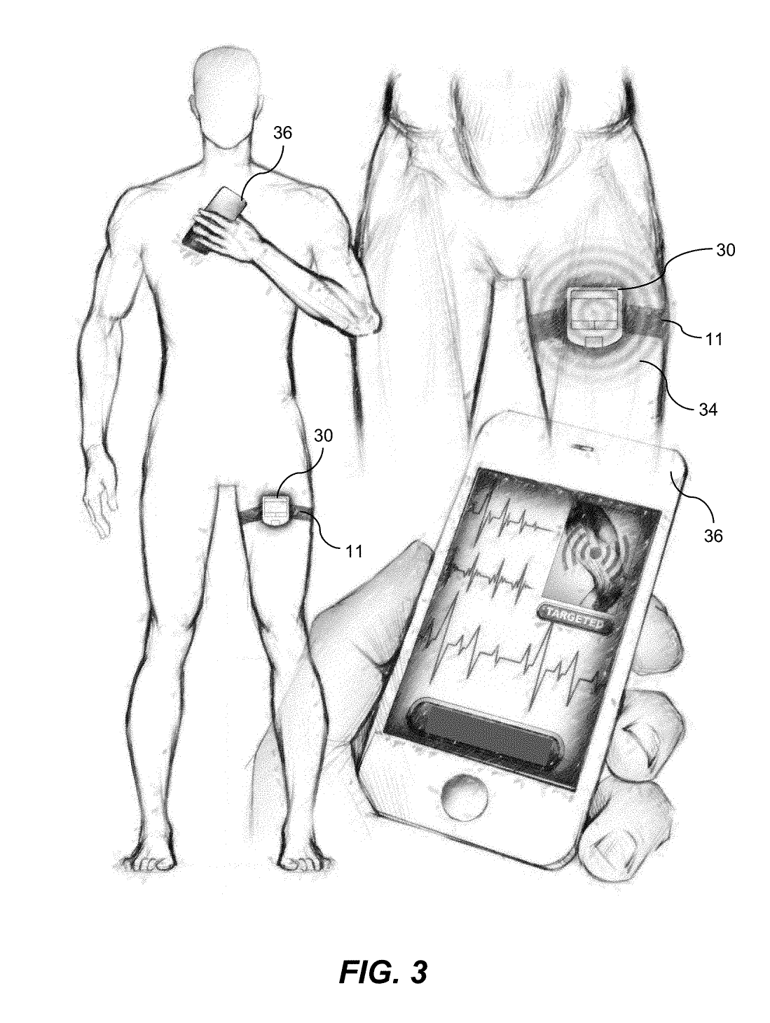

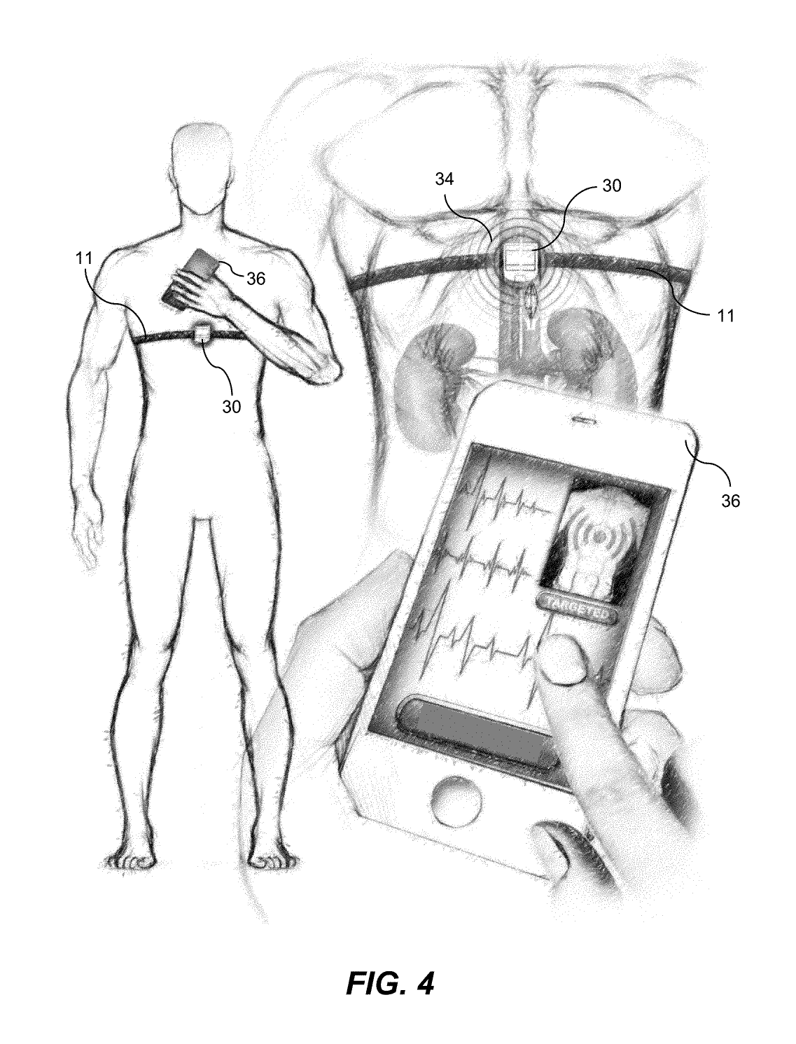

The device 30 (FIGS. 3 and 4) typically includes a sensor, an electromagnetic radiation generator or emitter, and a microprocessor analyzer optionally mounted or affixed, e.g., by a belt 11 to a subject. In some embodiments, it lacks (or disables or ignores) the sensor, and predetermined signals may then be used for application to the blood contact surface as desired. The electromagnetic radiation generator can be any device able to create and deliver and/or target electromagnetic radiation. The microprocessor analyzer can be any microprocessor with associated memory able to store information that can be retrieved on demand. The device includes an associated resonant calibration switch for triggering the device to scan, e.g., a blood contact device 32 to determine the highest risk stagnation points, and then to deliver a sequence of signals 34 at each stagnation point to prevent clot formation or calcification. The device 30 communications with, e.g., a subject or health care provider's cellular telephone 36, e.g., by BLUE TOOTH™ technology, instructing the subject, e.g., where to best position the belt for optimization of results at particular times Afterward, the operator may, e.g., then actuate a “run thrombosis prevention loop” program.

A preferred electromagnetic surface treatment device utilizes a custom electromagnetic signal for each surface stagnation point prone to blood clot formation, calcification, and/or plaque aggregation and stops the initial particles from adhering; thus stopping the progressive build up before it starts. Previous devices failed to tune to the correct frequency for the specific treatment surface. The instant device customs tunes to deter the beginning of buildup and blood clot formation, calcification, and/or plaque aggregation.

The device may then customize the electromagnetic signal for the specific treatment surface and cycles through a variety of signals when needed for multi-surface applications.

The electromagnetic surface treatment device preferably first non-invasively scans a blood contact device and determines the highest risk thrombosis points in a system or blood contact device

The electromagnetic surface treatment device then, starting with the highest risk location, delivers a succession of electromagnetic signals non-invasively for each stagnation high risk point of the blood contact device. This electromagnetic radiation tuning information is stored in a microprocessor preferably associated with the electromagnetic surface treatment device. The signals may then be delivered, based upon the stored information, in a loop from the signal generator (e.g., on a belt outside the patient), to each identified stagnation point, preferably in sequence from highest risk of thrombosis to lowest risk, which is again and again repeated.

By delivering electromagnetic energy to the blood contact device stagnation points, the initiation of thrombosis formation is prevented, thus preventing the accumulation of thrombosis to a dangerous risk level for stroke, pulmonary embolism, and/or other blood clot induced ailments. The device may be used to prevent blood clot(s), plaque(s), and/or calcification(s) on any blood contact surface, including living surfaces such as heart valves.

In manufacturing and testing, the device is first preferably mounted in a manner similar to the way it will be put to use with a subject.

There are a variety of techniques to apply an electromagnetic signal to the surface.

Potential applications for the device include applying electromagnetic radiation to heart implants, left ventricle assist devices (which are place closer to the heart), in place heart valves, and/or coronary arteries and coronary stents within coronary arteries that wrap around the heart, in the legs (see, e.g., FIG. 3, but also including having multiple devices up on down the legs separated about 12 inches apart from the upper thigh to the ankles).

The invention is further described with the aid of the following illustrative Examples.

### EXAMPLES

### Example I—Wireless Powered and Miniature Stent-Based Circulatory Assist Pumps

A wireless powered and stent based circulatory assist pump is made utilizing various technologies. For example, an Intravascular Miniature Stent Pump can be made as described in U.S. Pat. No. 7,998,190 (Aug. 16, 2011), the contents of which are incorporated herein by this reference. A Hydroimpedance Pump can be made as described in U.S. Pat. No. 7,163,385 (Jan. 16, 2007), the contents of which are incorporated herein by this reference. A Resonant Multilayer Impedance Pump can be made as described in U.S. Pat. No. 8,092,365 (Jan. 10, 2012), the contents of which are incorporated herein by this reference. A Helically Actuated Positive-Displacement Pump and Method can be made and used as described in U.S. Pat. No. 7,883,325 (Feb. 8, 2011), the contents of which are incorporated herein by this reference.

Using such technologies potentially reduces hemolysis, thrombosis, infection and mechanical breakdowns found in other circulatory assist pumps. Using these described technologies, a wireless powered circulatory assist pump is designed to be placed within the aorta of a patient. The product is designed to reduce the risk of hemolysis, thrombosis, mechanical breakdown, and infection associated with previously developed circulatory assist devices as well as easing placement.

This technology may be applied to the treatment of heart failure, reducing risk in high risk PCI, cardiogenic shock recovery, kidney protection, and limb salvage. One of the uses of the device is to eliminate excess fluid buildup from a patient suffering from heart failure. The wireless powered circulatory assist pump may have the option for both continuous and pulsatile flow.

To this technology is applied a wireless harmonic vibration technology, which reduces risk associated with thrombosis (blood clotting), often a problem associated with previous chronic long term use circulatory assist devices.

In certain embodiments, resonant frequency may be determined by placing the object next to a speaker (ex vivo) and also placing a microphone attached to an oscilloscope next to the object. The speaker plays a tone at a given volume, and then—without changing the volume—the pitch (or frequency) is slowly changed. Observing the oscilloscope identifies certain frequencies that the amplitude of the wave, which is proportional to the volume of the sound being picked up by the microphone, is greater than at surrounding frequencies. These are the resonant frequencies of the object, and are detectable as the sound energy absorbed by the object is re-emitted more efficiently at these pitches. The resulting data may then be entered into the device for application and use after the blood contact device has been implanted into the subject.

### Example II—Harmonic Vibration Device to Prevent Blood Clot Formation, Calcification and/or Plaque Formation on Blood Contact Surfaces

As previously described herein, blood clots, calcification and plaque form on blood contact surfaces within the human body and within blood contact medical devices. These unwanted build ups can lead to strokes, embolisms, and heart attacks, causing loss of life, brain or body functions and quality of life for a subject.

An instant harmonic vibration device that customs tunes at the exact right frequency to reach harmonic vibration of the particular stagnation prone surface thus preventing the beginning of buildup is made. The instant harmonic vibration device customizes a vibration signal for each surface stagnation point prone to blood clot formation, calcification, and/or plaque aggregation, and stops the first particles from adhering and thus stops the progressive build up before it starts.

The device prevents the starting point of buildup, and customizes the vibrational signal to reach harmonic frequency for the specific treatment surface and cycles through a variety of signals when needed for multi-surface applications. As such, the device can be used to stop clots, plaques, and/or calcification in any vessel not just blood contact devices. The device may also be used to lower blood pressure in patients. It may also be used to improve the general health of patients.

This exemplary device includes (a) a harmonic vibration signal generator and (b) a harmonic vibration microprocessor analyzer, both of which are essential for this device. The (a) harmonic vibration signal generator can be any device able to create and deliver acoustic or other vibrational energy signals. The (b) harmonic vibration microprocessor analyzer can be any microprocessor able to store information that can be retrieved on demand.

In use, the (a) harmonic vibration signal generator targets, one by one, the potential thrombosis stagnation points of the blood contact surface of a device, and delivers a succession of vibrational signals thereto. When the resonant frequency of a blood contact surface is reached, the (b) harmonic vibration microprocessor analyzer records this into its algorithm.

This process is repeated for all the stagnation points of the blood contact device, and this information is stored in the (b) harmonic vibration microprocessor analyzer. The sequence from highest risk to lowest risk is run in a loop by the (a) harmonic vibration signal generator with information stored in the (b) harmonic vibration microprocessor analyzer.

The (b) harmonic vibration microprocessor analyzer stores the information signals that directs the (a) the harmonic vibration signal generator to deliver the optimal signals to reduce risk of thrombosis at the blood contact device highest risk stagnation points.

In use, the operator may point or direct the (a) harmonic vibration signal generator—usually on a belt on the exterior of the patient—at the particular blood contact device. An associated resonant calibration switch is actuated, which triggers the device to scan the blood contact device, to determine the highest risk stagnation points, and then to deliver a sequence of signals at each stagnation point until resonant harmonic vibration is reached. This information may then be stored in an associated microprocessor of the device. After this is completed, the operator then actuates the run thrombosis prevention loop program, which program cycles the signal generator to deliver the resonant harmonic frequencies one by one to each high risk stagnation point.

### Example III—Wireless Powered and Miniature Stent-Based Circulatory Assist Pumps

A wireless powered and stent based circulatory assist pump is made utilizing various technologies. For example, an Intravascular Miniature Stent Pump can be made as described in U.S. Pat. No. 7,998,190 (Aug. 16, 2011), the contents of which are incorporated herein by this reference. A Hydroimpedance Pump can be made as described in U.S. Pat. No. 7,163,385 (Jan. 16, 2007), the contents of which are incorporated herein by this reference. A Resonant Multilayer Impedance Pump can be made as described in U.S. Pat. No. 8,092,365 (Jan. 10, 2012), the contents of which are incorporated herein by this reference. A Helically Actuated Positive-Displacement Pump and Method can be made and used as described in U.S. Pat. No. 7,883,325 (Feb. 8, 2011), the contents of which are incorporated herein by this reference.

Using such technologies potentially reduces hemolysis, thrombosis, infection and mechanical breakdowns found in other circulatory assist pumps. Using these described technologies, a wireless powered circulatory assist pump is designed to be placed within the aorta of a patient. The product is designed to reduce the risk of hemolysis, thrombosis, mechanical breakdown, and infection associated with previously developed circulatory assist devices as well as easing placement.

This technology may be applied to the treatment of heart failure, reducing risk in high risk PCI, cardiogenic shock recovery, kidney protection, and limb salvage. One of the uses of the device is to eliminate excess fluid buildup from a patient suffering from heart failure. The wireless powered circulatory assist pump may have the option for both continuous and pulsatile flow.

To this technology is applied the wired (or wireless) electromagnetic technology, which reduces risk associated with thrombosis (blood clotting), often a problem associated with previous chronic long term use circulatory assist devices.

### Example IV—Electromagnetic Surface Treatment Device to Prevent Blood Clot Formation, Calcification and/or Plaque Formation on Blood Contact Surfaces

As previously described herein, blood clots, calcification and plaque form on blood contact surfaces within the human body and within blood contact medical devices. These unwanted build ups can lead to strokes, embolisms, and heart attacks, causing loss of life, brain or body functions and quality of life for a subject.

An instant electromagnetic surface treatment device that customs tunes at the exact right frequency for the particular stagnation prone surface thus preventing the beginning of buildup is made. The instant electromagnetic surface treatment device customizes an electromagnetic signal for each surface stagnation point prone to blood clot formation, calcification, and/or plaque aggregation, and stops the first particles from adhering and thus stops the progressive build up before it starts.

The device prevents the starting point of buildup, and customizes the electromagnetic signal for the specific treatment surface and cycles through a variety of signals when needed for multi-surface applications. As such, the device can be used to stop clots, plaques, and/or calcification in any vessel not just blood contact devices. The device may also be used to lower blood pressure in patients. It may also be used to improve the general health of patients.

This exemplary device includes (a) an electromagnetic radiation generator and (b) a microprocessor analyzer, both of which are essential for this device. The (a) electromagnetic radiation generator can be any device able to create and deliver the selected electromagnetic radiation. The (b) microprocessor analyzer can be any microprocessor able to store information that can be retrieved on demand.

In use, the (a) electromagnetic radiation generator targets, one by one, the potential thrombosis stagnation points of the blood contact surface of a device, and delivers a succession of electromagnetic signals thereto. When the resonant frequency of a blood contact surface is reached, the (b) microprocessor analyzer records this into its algorithm.

This process is repeated for all the stagnation points of the blood contact device, and this information is stored in the (b) microprocessor analyzer. The sequence from highest risk to lowest risk is run in a loop by the (a) electromagnetic radiation generator with information stored in the (b) microprocessor analyzer.

The (b) microprocessor analyzer stores the information signals that directs the (a) the electromagnetic radiation generator to deliver the optimal signals to reduce risk of thrombosis at the blood contact device highest risk stagnation points.

In use, the operator may point or direct the (a) electromagnetic radiation generator—usually on a belt on the exterior of the patient—at the particular blood contact device. An associated resonant calibration switch is actuated, which triggers the device to scan the blood contact device, to determine the highest risk stagnation points, and then to deliver a sequence of signals at each stagnation point. This information may then be stored in an associated microprocessor of the device. After this is completed, the operator then actuates the run thrombosis prevention loop program, which program cycles the signal generator to deliver the appropriate wavelengths and frequencies one by one to each high risk stagnation point.

### REFERENCES

(The contents of the entirety of each of which is incorporated herein by this reference.)
Alexandrov et al., for the CLOTBUST Investigators. Ultrasound-Enhanced Systemic Thrombolysis for Acute Ischemic Stroke. N Engl J Med, 2004, 351, 2170-2178. DOI: 10.1056/NEJMoa041175.
A. Blitz “Pump thrombosis-A riddle wrapped in a mystery inside an enigma”, Ann Cardiothorac Surg. 2014 September; 3(5): 450-471; doi: 10.3978/j.issn.2225-319X.2014.09.10.
Daffertshofer et al. Transcranial low-frequency ultrasound-mediated thrombolysis in brain ischemia: increased risk of hemorrhage with combined ultrasound and tissue plasminogen activator: results of a phase II clinical trial. Stroke, 2005, 36(7), 1441-1446. DOI: 10.1161/01.STR.0000170707.86793.1a.
D. Grady “Sound Tested to Blast Heart Clots” New York Times (Mar. 19, 1997).
Grube et al. “High-frequency mechanical vibration to recanalize chronic total occlusions after failure to cross with conventional guidewires”, J Invasive Cardiol. 2006 March; 18(3):85-91.
Hoffmann & Gill, “Externally Applied Vibration at 50 Hz Facilitates Dissolution of Blood Clots In-Vitro”,Am. J. Biomed. Sci., 4(4):274-284 (2012); doi: 10.5099/aj120400274.
Hoffmann & Gill, “A study to determine chest wall vibratory attachment interface locations for a low frequency sonic vibrator in treatment of acute coronary thrombosis”, J Thromb Thrombolysis. 2011 August; 32(2):167-76; doi: 10.1007/s11239-011-0589-2.
Hudson et al. “Adjunctive transcutaneous ultrasound with thrombolysis: results of the PLUS (Perfusion by ThromboLytic and UltraSound) trial.” JACC Cardiovasc Interv. 2010, Mar., 3(3), 352-9. DOI: 10.1016/j.jcin.2009.11.020.
Jezovnik et al. “Medical complications in patients with LVAD devices” E-Journal of Cardiology Practice, 14(37) (European Society of Cardiology, 13 Feb. 2017).
Koiwa et al. “Precordial or epicardial input of phase controlled minute vibration: Effect on the coronary flow rate in regional ischemia.” New Horizons for Failing Heart Syndrome, 1996, pp. 117-130.
Koiwa et al. “Modification of Human Left Ventricular Relaxation by Small Amplitude, Phase—Controlled Mechanical Vibration on the Chest Wall.” Circulation, 1997, 95, 156-162. DOI: 10.1161/01 CIR.95.1.156.
Lee et al. “Computational analysis of blood clot dissolution using a vibrating catheter tip”, Proc Inst Mech Eng H. 2012 April; 226(4):337-40.
Ramachandran et al. “Vibrations and spatial patterns in biomimetic surfaces: using the shark-skin effect to control blood clotting”, Philos Trans A Math Phys Eng Sci. 2016 Aug. 6; 374(2073). pii: 20160133; doi: 10.1098/rsta.2016.0133.
Rame et al. “Unexpected Abrupt Increase in Left Ventricular Assist Device Thrombosis” N Engl J Med 2014; 370:1466-1467 Apr. 10, 2014; DOI: 10.1056/NEJMc1402425.
Gemma Reguera, “When microbial conversations get physical”, Trends Microbiol., 19(3): 105-113 (published online 2011 Jan. 14); doi: 10.1016/j.tim.2010.12.007.
Nakazawa et al. “Vibration assessment for thrombus formation in the centrifugal pump” Artif Organs. 1997 April; 21(4):318-22.
Slikkerveer et al. “Therapeutic application of ultrasound: contrast-enhanced thrombolysis in acute ST-elevation myocardial infarction; the Sonolysis study.” Neth Heart J. 2011 April; 19(4), 200-205. Published online 2011 March 8. DOI: 10.1007/s12471-011-0100-x.
Tosi et al. “Vibrational spectroscopy as a supporting technique in clinical diagnosis and prognosis of atherosclerotic carotid plaques: a review”, Anal Quant Cytopathol Histpathol. 2012 August; 34(4):214-32.
Tsiouris et al. “Short and long term outcomes of 200 patients supported by continuous-flow left ventricular assist devices”, World J Cardiol. 2015 Nov. 26; 7(11): 792-800 (published online 2015 Nov. 26); doi: 10.4330/wjc.v7.i11.792.
Yang et al. “Effect of pulse repetition frequency of high-intensity focused ultrasound on in vitro thrombolysis”, Ultrason Sonochem. 2017 March; 35(Pt A):152-160; doi: 10.1016/j.ultsonch.2016.09.014. (Epub 2016 Sep. 19).
Yohannes & Hoffmann “Non-invasive low frequency vibration as a potential emergency adjunctive treatment for heart attack and stroke. An in vitro flow model”, J Thromb Thrombolysis. 2008 June; 25(3):251-8. (Epub 2007 May 30).
U.S. Pat. No. 3,352,303 to Delaney describes a method for blood clot lysis by the direct application to the clot of sonic or supersonic energy vibrations.
U.S. Pat. No. 4,058,075, which issued to Piper on Nov. 15, 1977, discloses a marine life growth inhibitor device. The device is used for inhibiting marine life on the outer surface of submerged object such as boat. The device includes a controller connected to a source of electrical power and a plurality of speakers electrically connected to the controller and attached at predetermined locations on the interior of the boat's hull, whereby vibrations may be transmitted through the hull.
U.S. Pat. No. 5,143,011 to Rabbette (Sep. 1, 1992) discloses a method and apparatus for inhibiting barnacle growth on boats. The system for inhibiting growth of barnacles and other marine life on the hull of a boat includes a plurality of transducers or vibrators mounted on the hull and alternately energized at a frequency of 25 Hertz through a power source preferably the boat battery, and a control system
U.S. Pat. No. 5,386,397 to Urroz (Jan. 31, 1995) describes method and apparatus for keeping a body surface, which is in contact with water, free of fouling. A sound wave is generated for keeping a surface free of scale, fouling and dirt by the adherence of organisms such as marine life, the surface being part of the body that is in contact with water.
U.S. Pat. No. 5,532,980 to Zarate, et al. (Jul. 2, 1996), discloses a vibrational anti-fouling system that produces vibrations in an underwater structure for the purpose of inhibiting the attachment of aquatic life forms to the structure. The system utilizes loudspeaker-like resonators which operate underwater and produce acoustic vibrations continually having a duty cycle of 3.65 seconds which includes a current drive period of 0.4 seconds.
WO01/58750 provides an ultrasonic anti-fouling device which teaches of devices that are hung outside a hull and arranged so that the vibrations are directed to run parallel to a surface of the hull.
U.S. Pat. No. 5,713,848 to Dubrul et al., Feb. 3, 1998, describes a catheter suitable for introduction into a tubular tissue for dissolving blockages in such tissue. It is useful for removing thrombi within blood vessels. Dissolution of vascular thrombi is facilitated by advancing a catheter through an occluded vessel, the catheter causing a vibrating, stirring action in and around the thrombus usually in combination with the dispensing of a thrombolytic agent (e.g., urokinase) into the thrombus. Catheters allow both low frequency (1-1000 Hz) vibratory motion and delivery of biological agents to a blockage.
U.S. Pat. No. 6,285,629 provides an ultrasonic vibration device; a voltage is applied to a submerged marine structure to exert thereon vibrational and electric energies, thereby effecting deterioration prevention of the structure. An ultrasonic vibration unit comprises an ultrasonic vibrator made from a piezoelectric ceramic plate with an electrode on each side thereof; power supply wires connected to the respective electrodes; a support member for fixedly supporting the ultrasonic vibrator and transmitting the ultrasonic vibration to the structure; and a resin coat for protecting the ultrasonic vibrator against seawater. The ultrasonic vibration unit is used for preventing deterioration of the submerged marine structure.
U.S. Pat. No. 7,517,328 to Hoffmann (Apr. 14, 2009) describes a low frequency vibration assisted blood perfusion emergency system.
US Patent Application Publication 2009/0314193 to Groves et al. (Dec. 24, 2009) describes an ultra-sonic device that inhibits growth of waterborne flora and fauna on, e.g., boats and the like.

What is claimed is:

1. A device comprising:
a sensor that detects harmonic frequency and/or electromagnetic energy and recognizes resonance and/or ionic charge on a blood contact surface,
a microprocessor for analyzing data from the sensor, and
an emitter of sound, ultrasound, and/or electromagnetic energy associated with said microprocessor that can create and focus a specific sound, ultrasound, and/or electromagnetic energy onto the blood contact surface.

2. The device of claim 1, wherein the sensor of the device detects harmonic frequency, and the device first reads and then the microprocessor custom tunes in the appropriate harmonic frequency to prevent the beginning of blood clot formation, calcification, and/or plaque formation on the blood contact surface.

3. The device of claim 2, wherein the emitter consists of a harmonic vibration signal generator and the microprocessor consists of a harmonic vibration microprocessor analyzer.

4. The device of claim 1, further comprising a strap that affixes the device to a living subject in which the blood contact surface has been placed.

5. The device of claim 1, further comprising an associated resonant calibration switch for triggering the device to scan a blood contact device having the blood contact surface to determine the highest risk stagnation point(s).

6. The device of claim 5, wherein the device delivers a sequence of signals to each stagnation point until resonant harmonic vibration for the blood contact surface is reached so that blood clots do not affix to the blood contact surface.

7. The device of claim 1, wherein the emitter emits electromagnetic radiation that applies a selected electromagnetic radiation to the blood contact surface, and
the microprocessor controls delivery of the electromagnetic radiation from the emitter to the blood contact surface so as to prevent the beginning of blood clot formation, calcification and/or plaque formation on the blood contact surface.

8. A method of using the device of claim 1, comprising:
applying an appropriate harmonic frequency or electromagnetic energy to a blood contact surface within a living subject so as to prevent and/or dislodge blood clot(s), calcification(s), and/or plaque formation(s) on the blood contact surface.

9. The method according to claim 8, wherein the appropriate harmonic frequency or electromagnetic energy is applied to the blood contact surface while not in physical contact with the structure.

10. The method according to claim 8, further comprising:
cycling through a variety of signals for a plurality of blood contact surfaces on a blood contact device.

11. A method of preventing the formation of a blood clot, calcification, and/or plaque on a blood contact surface, the method comprising:
applying a selected harmonic frequency or electromagnetic energy to a blood contact surface to attain resonance and prevent the beginning formation of a blood clot formation, calcification, and/or plaque formation on the blood contact surface.

12. The method according to claim 11, wherein a wireless harmonic-tuned vibration device applies a harmonic frequency to the blood contact surface.

13. The method according to claim 12, wherein the wireless harmonic-tuned vibration device has a custom signal for each of various multiple high risk stagnation points on a blood contact device.

14. The method according to claim 11, further comprising recording the signals ex vivo for reaching resonance for each stagnation point on the blood contact surface.

15. The method according to claim 11, further comprising playing back the signals in cycles on loop.

16. The method according to claim 11, wherein the method comprises:
measuring an appropriate harmonic frequency to prevent the beginning formation of a blood clot formation, calcification, and/or plaque formation on a blood contact surface of a device; and
custom tuning and applying the appropriate harmonic frequency to the blood contact surface to prevent the initial formation of a blood clot formation, calcification, and/or plaque formation on the blood contact surface.

17. The method according to claim 16, wherein the wireless harmonic-tuned vibration device has a custom signal for multiple high risk stagnation points on the blood contact surface.

18. The method according to claim 11, further comprising:
recording the signals for reaching resonance for each stagnation point on the blood contact surface.

19. The method according to claim 11, wherein the blood contact surface is part of a device selected from the group consisting of a heart valve, pacemaker, and a left ventricle assist device.
